(19) United States
(12) Patent Application Publication
(10) Pub. No.: US 2011/0230786 A1
(43) Pub. Date: Sep. 22, 2011

(54) HEARING TESTING DEVICE

(76) Inventor: Philip Stuart Esnouf, Richmond (AU)

(21) Appl. No.: 13/051,925

(22) Filed: Mar. 18, 2011

Related U.S. Application Data
(63) Continuation-in-part of application No. PCT/AU2009/001241, filed on Sep. 18, 2009.
Application No. PCT/AU2009/001241 is a continuation-in-part of application No. 11/547,585, filed on Sep. 30, 2008, now abandoned.

(30) Foreign Application Priority Data
Apr. 8, 2004 (AU) 2004901920
Sep. 18, 2008 (AU) 2008904875

(51) Int. Cl. A61B 5/00

(52) U.S. Cl. 600/559

(57) ABSTRACT

A portable wireless hearing testing device, the device including: left and right earpieces connected together by a headband and arranged as headphones; the left and right earpiece each including an earpiece housing shaped to surround the left and right ears of a subject respectively to substantially prevent ambient sounds reaching the ear of the subject; left and right loudspeakers located within the left and right earpiece housings respectively; and control circuitry located within the left and/or right earpiece housings and coupled to drive the loudspeakers, the control circuitry being arranged to produce left and right test signals which are applied to the left and right loudspeakers respectively, the test signals being operative to cause the left and right loudspeakers to provide a respective series of tones at frequencies corresponding to the test signals and wherein the test signals do not include significant harmonic components of the tones.

### BACKGROUND

1. Technical Field

This disclosure relates to an improved hearing testing device and method of assessing hearing impairment of a subject.

2. Description of the Related Art

International Publication No. WO 2005/096940 discloses a hearing testing device which is portable and can be used to produce a series of tones at predetermined frequencies and at a predetermined volume in order to screen patients for initial assessment of possible hearing impairment.

### BRIEF SUMMARY

Whilst the aforementioned device is very satisfactory, it does have the drawback that only a single ear of the subject is tested at a time. This can cause difficulties in obtaining an accurate screening where there is ambient noise because the ear which is not being tested is subject to ambient noise.

The aforementioned device also has the drawback that the subject or operator needs to physically move the device from one ear to another and the operator and/or subject need to move in order that the operator can visually observe the indicators on the screening device.

Some embodiments of the disclosure provide a hearing testing device and method which at least partially overcomes some of the disadvantages of the aforementioned device.

According to the present disclosure there is provided a portable wireless hearing testing device, the device including: left and right earpieces connected together by a headband and arranged as headphones; the left and right earpiece each including an earpiece housing shaped to surround the left and right ears of a subject respectively to substantially prevent ambient sounds reaching the ear of the subject; left and right loudspeakers located within the left and right earpiece housings respectively; and control circuitry located within the left and/or right earpiece housings and coupled to drive the loudspeakers, the control circuitry being arranged to produce left and right test signals which are applied to the left and right loudspeakers respectively, the test signals being operative to cause the left and right loudspeakers to provide a respective series of tones at frequencies corresponding to said test signals and wherein the test signals do not include significant harmonic components of said tones.

Preferably, the control circuitry is arranged to produce all of the left or right signals automatically followed by all of the right or left test signals.

Preferably further, the device includes left and right visual indicating means, there being separate left indicators for each tone frequency and separate right indicators for each tone frequency. Tones generated in the right ear are indicated by LEDs that illuminate red, tones generated in the left ear are indicated by LEDs that illuminate blue, this is in keeping with Audiology convention.

Preferably further, the control means is operable to cause the left indicators to be operative in synchronism with the respective tone frequencies produced by the left loudspeaker and is operable to cause the right indicators to be operative in synchronism with the respective tone frequencies produced by the right loudspeaker.

Preferably further, the operative period of the indicators has the same duration as the respective tone frequencies.

Preferably further, the left and right indicators comprise LEDs mounted on a circuit board which is located within the left or the right earpiece housing and wherein the left or right earpiece housing includes openings to enable viewing by an operator of all of said LEDs.

Preferably further, the left or right earpiece includes indicia to identify the respective tone frequencies for the left and right indicators.

Preferably further, the tone frequencies are IkHz₅ 2 kHz, 4 kHz and 6 kHz.

Preferably the tone frequencies are accurate to within the range ±2%.

Preferably further, the loudspeakers generate said tones at 25±1 dBHL.

Preferably further, the total harmonic distortion is less than 1% and most preferably is about 0.1%.

The disclosure also provides a method of assessing hearing impairment of a subject including the steps of locating left and right earpieces, which are connected together by a headband over the left and right ears respectively of a subject; actuating a switch which is coupled to activate control circuitry located within the left and/or right earpiece to generate a predetermined series of tones which do not include significant harmonic components of said tones in the left and right earpieces and, simultaneously activating indicating means when the respective tones are being produced.

### BRIEF DESCRIPTION OF THE SEVERAL VIEWS OF THE DRAWINGS

The disclosure will now be further described with reference to the accompanying drawings, in which: FIG. 1 is a schematic view of an improved hearing device of the disclosure;

FIG. 2 is a fragmentary side view of the left earpiece;

FIG. 3 is a fragmentary view of the inner side of the left earpiece;

FIG. 4 is a schematic view of the left earpiece with the inner part removed;

FIG. 5 is a fragmentary cross-sectional view along the line 5-5;

FIG. 6 is a schematic side view of the inner part of the left earpiece;

FIG. 7 is a block diagram illustrating the function of the control circuitry;

FIG. 8 are waveform diagrams illustrating the function of the circuitry; and

FIG. 9 is a circuit diagram for the control circuitry.

### DETAILED DESCRIPTION

FIG. 1 shows an improved hearing testing device 2 constructed in accordance with the disclosure. The drawing shows a rear view of the device 2 having a left earpiece 4, right earpiece 6 connected together by means of a resilient headband 8 which includes a padded central portion 10. The left earpiece 4 includes a hollow housing assembly 12 which is fitted with a padded ring 14. Similarly, the right earpiece 6 includes a right earpiece assembly 16 fitted with a padded ring 18. Except for tones generated by loudspeakers (not shown in FIG. 1) located within the device, the earpieces 4 and 6 effectively isolate the wearer from ambient noise. It will be seen that the earpieces 4 and 6 are pivotally connected to yokes 20 and 22 which form part of the headband 8. The yokes 20 and 22 are connected by means of swivel connections 24 and 26 to the headband so as to arrange biaxial rotation of each of the earpieces 4 and 6.

In the illustrated arrangement, the left earpiece 4 includes control circuitry 100 for producing tones in both of the earpieces 4 and 6. It will be appreciated, of course, that the control circuitry could be located in the right earpiece.

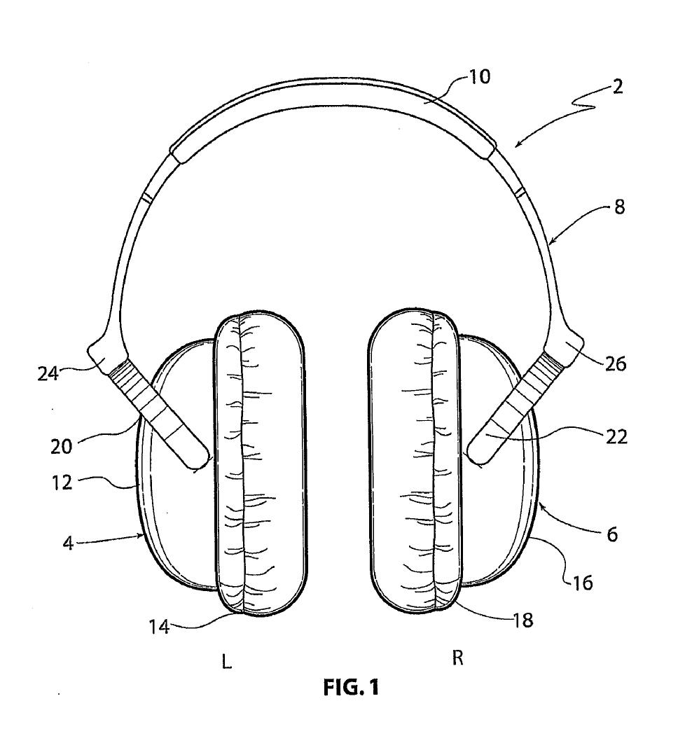

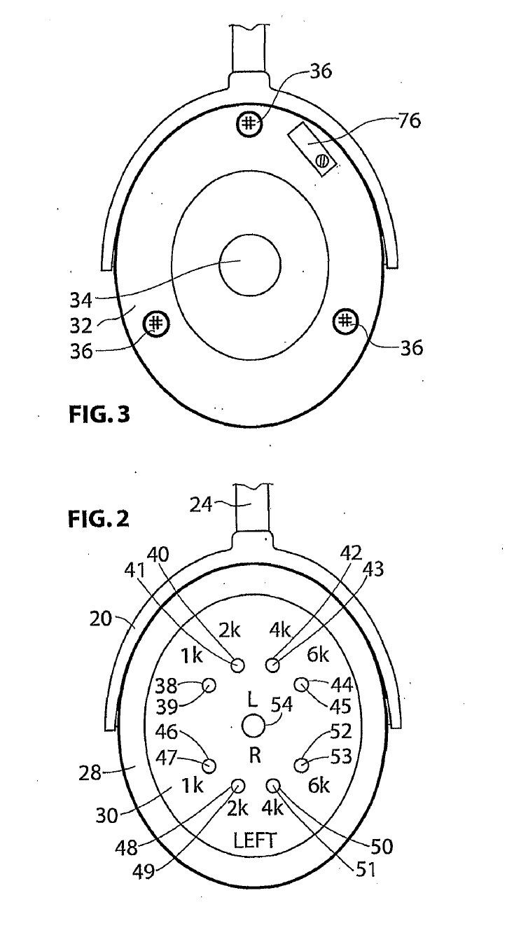

FIGS. 2 and 3 show the outside and inside faces respectively of the left earpiece 4 with the padded ring 14 removed for clarity of illustration. The housing assembly 12 includes a main hollow body 28 and a central body 30 which together form a cup shape having its opening facing inwardly. The left earpiece 4 includes an inner cover 32 which serves as the mounting for the left loudspeaker 34. In the illustrated arrangement, the cover 32 is connected to the housing assembly 12 by means of three screws 36.

The central body 30 includes various openings to provide access to the control circuitry located within the housing assembly 12. More particularly, the central body 30 includes four openings 38, 40, 42 and 44 which enable a user to see four left LEDs 39, 41, 43 and 45 located beneath the openings. The central body 30 also includes a further four openings 46, 48, 50 and 52 to enable an operator to see four right LEDs 47, 49, 51 and 53 located beneath the openings. The central body 30 further includes a central opening through which a press button switch 54 projects. The central body 30 is also printed with the word LEFT to indicate that it is the left earpiece. It also has printed thereon indicia which indicate the frequency being generated when corresponding LED devices are activated. In the illustrated arrangement, the indicia 1 kHz, 2 kHz, 4 kHz and 6 kHz are printed adjacent to the openings 38, 40, 42 and 44 respectively and the letter L shows that the LEDs are activated when the left; loudspeaker 34 produces tones. Similar indicia are provided for the openings 46, 48, 50 and 52 for the right loudspeaker. It will be appreciated, of course, that it would be possible to rearrange the location of the LEDs and achieve similar results. For instance, the right LEDs could be located above the left. Further, in the illustrated arrangement all of the LEDs and the press button switch 54 are on the left earpiece 4 but they could alternatively be provided on the right earpiece 6.

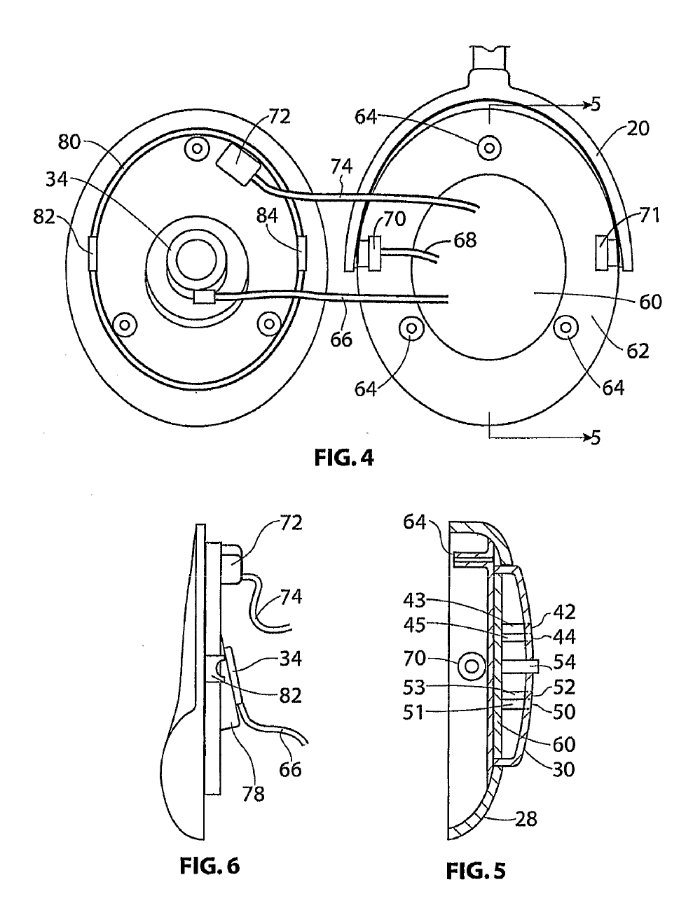

FIGS. 4, 5 and 6 show further details of the left earpiece 4 again with the padded ring 14 removed. In FIG. 4 the inner cover 32 has also been removed to reveal the location of the internal components. The control circuitry 100 includes a circuit board 60 which is located within the housing assembly 12 and retaining position by a retaining plate 62. The retaining plate 62 is formed with three screw posts 64 which receive the mounting screws 36. It will be seen that the circuitry includes left loudspeaker wires 66 which connect the circuitry to the left loudspeaker 34. The circuit also includes right loudspeaker wires 68 for connecting the circuitry to the right loudspeaker 35 (see FIG. 9). In the illustrated arrangement, the right loudspeaker wire 68 extends through a hollow shaft 70 which forms part of the pivotal connection of the yoke 20 to the housing assembly 12. A hollow shaft 71 forms the pivotal connection at the other side of the assembly 12. A battery 72 is mounted on the inside of the cover 32 and is connected to the circuit board 60 by means of battery leads 74. The battery is changeable by removing a battery cover 76 which is located on the outer face of the cover 32 as shown in FIG. 3. The inside face of the cover 32 is formed with a central platform 78 which provides a preferred orientation of the loudspeaker 34 for directing sound waves produced thereby into the ear canal of the wearer. The inside face of the cover 32 also includes a flange wall 80 which serves to impart rigidity to the cover. The flange wall 80 includes two brackets 82 and 84 which include semi-circular recesses which engage the shafts 71 and 70 respectively in order to stabilise the pivotal connections of the yoke 20 to the earpiece 4.

It will be seen from FIG. 5 that the circuit board 60 includes the left and right LEDs mounted thereon and these are located beneath the respective openings in the central body 30. It will also be seen that the press button switch 54 projects from switch components located on the circuit board 60.

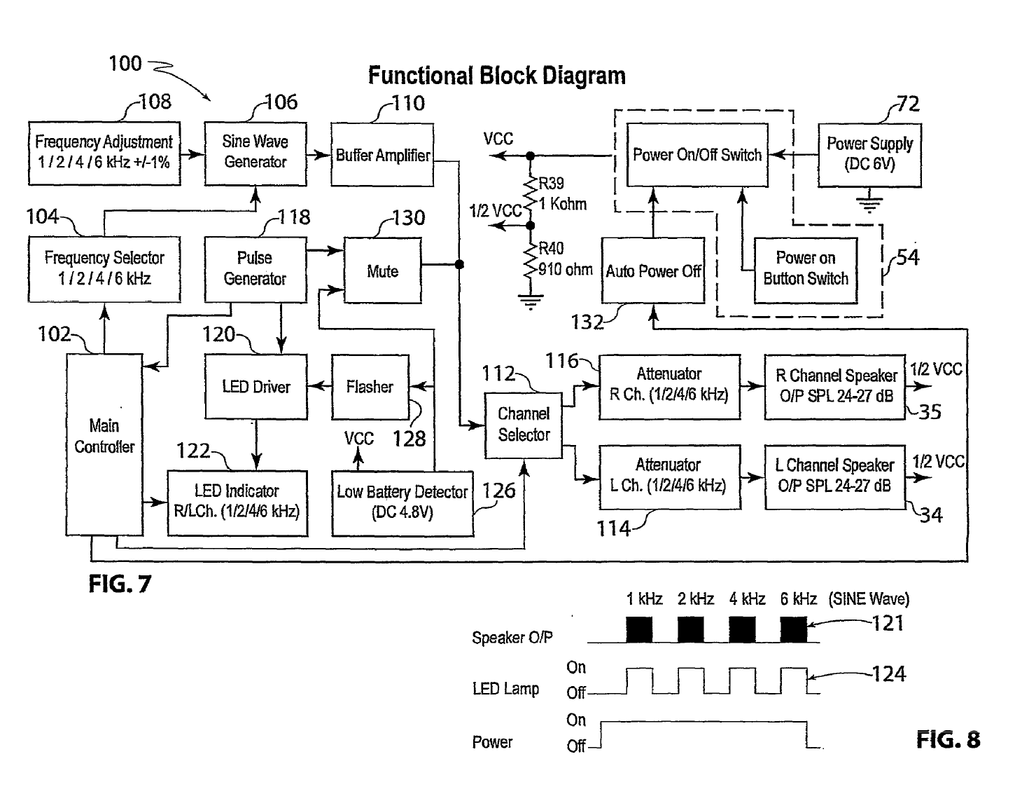

FIG. 7 is a block diagram showing the control circuitry 100 in more detail. It will be seen that the battery 72 is coupled via the press button switch 54 to provide power to other components in the circuitry. Once the press button switch 54 has been applied, power is supplied to a main controller 102. The main controller 102 has an output coupled to a frequency selector circuit 104, the output of which is in turn connected to a sine wave generator 106 which is thereby controlled so as to selectively produce signals at 1 kHz, 2 kHz, 4 kHz and 6 kHz. The circuit includes a frequency adjustment circuit 108 which operates to accurately control the frequencies generated by the sine wave generator 106. Output from the sine wave generator 106 is connected to a buffer amplifier 110 which in turn is connected to a channel selector circuit 112. Output from the channel selector circuit 112 is then connected to the left loudspeaker 34 via an attenuator 114 and to the right loudspeaker 35 via an attenuator 116.

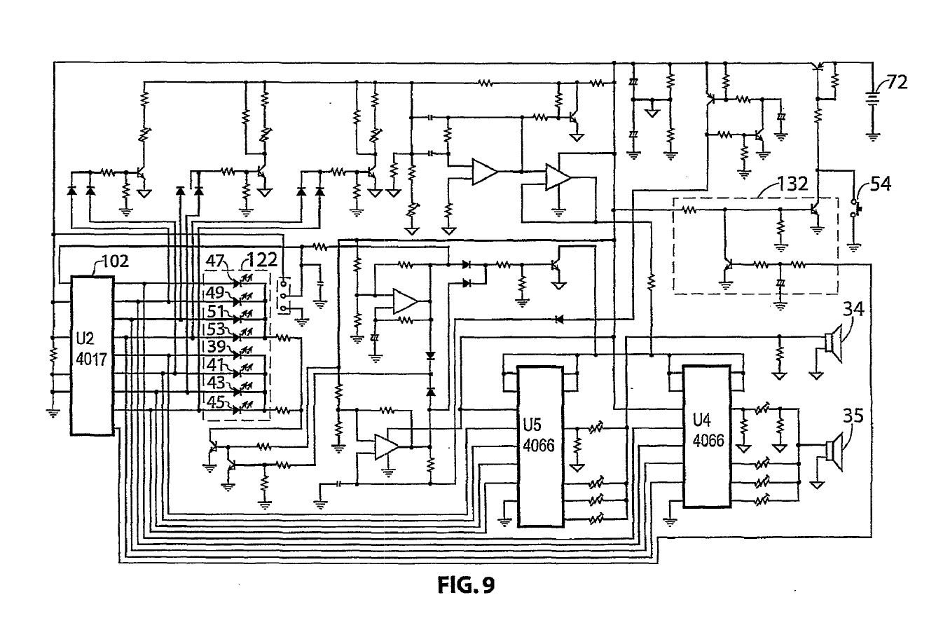

The control circuitry 100 includes a pulse generator 118 which controls the duration and spacing of the tone pulse as indicated by the waveform 120 of the diagram of FIG. 8. Output from the pulse generator 118 is coupled to an input of the main controller 102 in order to blank out signals therefrom for controlling the sine wave generator 106 to thereby create the bursts of tones, as required. Output from the pulse generator 118 is also connected to LED driver circuits 121 which are coupled to the indicator LEDs collectively shown as block 122. Waveform 124 shows output from the LED driver circuit 121 (which is the same as the waveform 120 of the LEDs). It will be seen that the LEDs are activated in synchronism and for the same duration as the speakers, as indicated by the waveforms 120 and 124.

The illustrated circuit includes a low battery detector circuit 126 which monitors the output of the battery 72 and when it falls below a predetermined level (say 4.8 volts where the battery 72 has a nominal rating of 6 volts), the circuit produces an output which is coupled to a flasher circuit 128 which in turn is coupled to the LED driver circuit 120.

The flasher circuit 128 thus causes the LEDs to flash when the battery is low, thereby indicating to the operator that the batteries need replacement. The illustrated circuit includes a muting circuit 130 which is coupled to the low battery detector circuit 126 and operates to deactivate the buffer amplifier 110 when a low battery is detected to thereby prevent tone signals being applied to the loudspeakers.

The circuit further includes an auto power OFF circuit 132 which is coupled to the main controller 102 and is operable to act on the switch 54 after all of the tones have been generated for the left and right loudspeakers. This conserves battery life.

The control circuitry 100 can be implemented in many ways as would be understood by a person skilled in the art. FIG. 9 shows one circuit implementation for the control circuitry 100. The detailed operation of this circuit need not be described in detail as it would be understood by a person skilled in the art.

In operation the operator or the user fits the device to the subject so that the earpieces 4 and 6 cover the left and right ears respectively of the subject. After fitting of the device, the subject is directed to indicate, say by moving his or her hand or finger, when a sound can be perceived. The operator is in a position whereby the LEDs can be monitored. The subject or the operator then presses the press button switch 54 in order to initiate the sequence. In the illustrated arrangement the right loudspeaker 35 is first activated so as to produce tones at IkHz, 2 kHz, 4 kHz and 6 kHz as indicated by the waveform 120. The right hand LEDs 47, 49, 51 and 53 are activated in synchronism with the tones. The operator can then determine which tones are perceived by the subject by monitoring the right hand LEDs at the various frequencies for the right ear. The left ear can be screened in a similar way. If the results are poor, the subject can be referred to an audiologist for clinical assessment for hearing impairment in the normal way. It will be appreciated that the device and method of the disclosure provide a very convenient form of initial screening of hearing impairment.

The improved hearing testing device and method of the disclosure provide a number of advantages over prior art devices. First, because there are earpieces for both ears, the problem of inaccurate readings caused by ambient noise is substantially reduced. Second, the device only needs to be applied once to the subject in order to test both ears. Also, the indicator LEDs are observed from the left hand earpiece and therefore once the device has been fitted to a subject, the subject and/or the operator does not need to move in order to monitor the LEDs and the subject's responses to tones for both ears. The auto power OFF conserves battery life. The low battery detector also avoids false readings being obtained when the batteries are run down. It will be appreciated that in a modified arrangement the mounting of the indicator LED's could be such that the left hand LED's are in the left earpiece and the right hand LED's are in the right earpiece and preferably located towards the front side so that the operator could view all of the LED's whilst facing the subject.

The circuit implementation of FIG. 9 has a number of advantages including low operating voltage (6 volts). It also has low power consumption enabling 700 cycles to be obtained using two CR3022 lithium coin batteries. The circuit has zero standby current. It produces very pure sinusoidal output having a distortion less that 0.01%. Potentiometers are included which can be adjusted if necessary to accurately control the level of sound to 25 dB plus or minus 1 dB and to fine tune the frequencies.

Many modifications will be apparent to those skilled in the art without departing from the spirit and scope of the disclosure.

The various embodiments described above can be combined to provide further embodiments. All of the U.S. patents, U.S. patent application publications, U.S. patent applications, foreign patents, foreign patent applications and non-patent publications referred to in this specification are incorporated herein by reference, in their entirety.

Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A portable wireless hearing testing device, the device including:
left and right earpieces connected together by a headband and arranged as headphones;
the left and right earpiece each including an earpiece housing shaped to surround the left and right ears of a subject respectively to substantially prevent ambient sounds reaching the ear of the subject;
left and right loudspeakers located within the left and right earpiece housings respectively; and
control circuitry located within the left and/or right earpiece housings and coupled to drive the loudspeakers, the control circuitry being arranged to produce left and right test signals which are applied to the left and right loudspeakers respectively, the test signals being operative to cause the left and right loudspeakers to provide a respective series of tones at frequencies corresponding to said test signals and wherein the test signals do not include significant harmonic components of said tones.

2. A device as claimed in claim 1 wherein the control circuitry is arranged to produce all of the left or right signals automatically followed by all of the right or left test signals.

3. A device as claimed in claim 1 including left and right visual indicating means, there being separate left indicators for each tone frequency and separate right indicators for each tone frequency.

4. A device as claimed in claim 3 wherein the control circuitry is operable to cause the left indicators to be operative in synchronism with the respective tone frequencies produced by the left loudspeaker and is operable to cause the right indicators to be operative in synchronism with the respective tone frequencies produced by the right loudspeaker.

5. A device as claimed in claim 4 wherein the operative period of the indicators has the same duration as the respective tone frequencies.

6. A device as claimed in claim 3 wherein the left and right indicators comprise LEDs mounted on a circuit board which is located within the left or the right earpiece housing and wherein the left or right earpiece housing includes openings to enable simultaneous viewing by an operator of all of said LEDs from one side of the device.

7. A device as claimed in claim 6 wherein the left or right earpiece includes indicia to identify the respective tone frequencies for the left and right indicators.

8. A device as claimed in claim 1 wherein the tone frequencies are 1 kHz, 2 kHz, 4 kHz and 6 kHz.

9. A device as claimed in claim 8 wherein the control circuitry is operable to generate said tone frequencies with an accuracy of ±2%.

10. A device as claimed in claim 1 wherein the total harmonic distortion in said test signals is less than 1%.

11. A device as claimed in claim 1 wherein the loudspeakers generate said tones at 25±1 dBHL.

12. A device as claimed in claim 1 including a battery coupled to power said control circuitry, the battery being located within one of said earpiece housings.

13. A method of assessing hearing impairment of a subject, comprising:
locating left and right earpieces, which are connected together by a headband over the left and right ears respectively of a subject; and
actuating a switch which is coupled to activate control circuitry located within the left and/or right earpiece to generate a predetermined series of tones which do not include significant harmonic components of said tones in the left and right earpieces; and
simultaneously activating indicating means when the respective tones are being produced.

14. A method as claimed in claim 13 including locating the indicating means such that they can be observed by an operator from a single observation position.

15. A method as claimed in claim 13 including providing a battery within one of the earpieces so that the earpieces and control circuitry are fully self-contained and do not require any wire or cable connection thereto.

16. A method as claimed in claim 13 wherein there are left indicators which operate in synchronism with respective tone frequencies produced by a left loud speaker and right indicators which operate in synchronism with the respective tone frequencies produced by a right loud speaker.
